(19) United States
(12) Patent Application Publication
(10) Pub. No.: US 2012/0213904 A1
(43) Pub. Date: Aug. 23, 2012

(54) NOVEL PANDAN EXTRACT ARTICLES IN POWDER FORM AND PROCESS FOR THE PRODUCTION THEREOF

(75) Inventors: Andreas Degenhardt, Holzminden (DE); Gerhard Krammer, Holzminden (DE); Jens Koch, Eschershausen (DE); Mee Yin Tai, Singapore (SG)

(73) Assignee: SYMRISE AG, Holzminden (DE)

(21) Appl. No.: 13/402,254

(22) Filed: Feb. 22, 2012

Related U.S. Application Data
(60) Provisional application No. 61/445,214, filed on Feb. 22, 2011.

(30) Foreign Application Priority Data
Feb. 22, 2011 (EP) 11155425.9

(51) Int. Cl. A23L 1/226; C07D 207/20; A23D 9/007; A23L 1/10; A23L 1/28; A21D 2/24

(52) U.S. Cl. 426/537; 426/655; 426/549; 426/601; 426/618; 548/540

(57) ABSTRACT

The present invention relates to novel pandan extract articles in powder form, wherein the artice comprises high amounts of precursors of 2-acetyl-1-pyrroline, a novel process of preparing such pandan extract articles in powder form and corresponding methods of using said pandan extract articles.

The present invention relates to a novel pandan extract article in powder form, wherein the article comprises advantageously high amounts of precursors of 2-acetyl-1-pyrroline. Said article is obtainable by a process described hereinafter.

The present invention thus also relates to a corresponding process of preparing a pandan extract article in powder form, preferably a pandan extract article according to the present invention, the process comprising the following steps:
providing pandan leaves or parts thereof,
contacting the pandan leaves or the parts thereof with water having a temperature in the range of from 5° C. to 40° C., so that an aqueous extract of ingredients of pandan leaves is obtained,
removing water from the aqueous extract, in the presence or absence of a carrier material or other additives, wherein the water is removed by spray drying the aqueous extract using an inlet temperature in the range of from 140 to 170° C., preferably in the range of from 150 to 160° C., and an outlet temperature below 80° C., preferably an outlet temperature in the range of from 70 to 75° C.,
   so that the pandan extract article in powder form is obtained.

The present invention further provides a novel method of imparting or enhancing organoleptic properties, the organoleptic properties being selected from the group consisting of mouthfullness and roasted notes, wherein a pandan extract article according to the invention or a pandan extract article prepared by a process according to the invention, respectively, is used.

Furthermore, the present invention relates to a method of masking an off-note of an edible fat or oil, wherein a pandan extract article according to the present invention or a pandan extract article prepared by a process according to the present invention is used.

The present invention also relates to a method of making edible food products and the use of a pandan extract article according to the present invention or prepared according to a process according to the present invention for
imparting or enhancing organoleptic properties, the organoleptic properties being selected from the group consisting of mouthfullness and roasted notes,
   and/or
masking off-notes of edible fats and oils.

Natural roast aromas are widely used in roasted food products such as coffee, malt, cocoa and the like. Natural roast aromas usually contain flavors depending on Maillard chemistry based on sugars and (free) amino acids as educts. The aroma profiles of such mixtures vary depending on said educts, further reaction partners or other compounds and the respective reaction conditions.

Especially aroma extracts of roasted coffee, which may be used as natural roast aroma in roasted food products, are rather unstable and difficult to obtain. It is therefore highly desirable to have an alternative source for roast aromas, preferably for natural roast aromas (i.e. from natural sources), which provides a more stable and more pleasant roasted character.

The use of (natural) roasted aromas or aroma extracts therefore, respectively, is wide-spread ranging from release of roasted notes for heated food products, such as soups, up to the use in cold applications, such as ice-cream, puddings, and desserts.

Pandanus amaryllifolius is a tropical plant which is commonly known as “pandan”. Pandan is widely used in south-east Asian cooking as a flavoring. In particular, the leaves of pandanus amaryllifolius are used either fresh or wilted and provide a nutty, botanical fragrance. As commonly believed, the characteristic aroma of pandan is mainly caused by the aroma compound 2-acetyl-1-pyrroline, which, inter alia, gives jasmine rice and basmati rice their typical smell.

2-Acetyl-1-pyrroline can be formed by Maillard reactions during heating of food such as in baked bread. In particular, as already known (see J. agric. Food Chem. 1998, 46, 616-619), 2-acetyl-1-pyrroline may be formed from ornithin, prolin or glutamic acid in Maillard reaction. However, it is presumed that 2-acetyl-1-pyrroline is not a direct reaction product of ornithin, prolin or glutamic acid, but merely of an intermediate product, i.e. there are unknown (direct) precursors of 2-acetyl-1-pyrroline.

Various alkaloids have been described for pandan (see e. g. J. Nat. Prod. 2004, 67, 54-57; ACGC Chemical research Communications, 1996, 5, 24-27, Phytochemistry, 1993, 34, 1159-1163; Chem. Pharm. Bull. 2002, 50, 1303-1304; J. Nat. Prod. 2001, 64, 1579-1580; J. Nat. prod. 2001, 64, 1224-1225; J. Am. Chem. Soc. 2000, 122, 8635-8639; Tetrahedron Letters 2001, 42, 2995-2996). However, from these papers it is unclear how roasted notes are formed. However, biosynthetic routes for the major roasted smelling compound from pandan, i.e. 2-acetyl-1-pyrroline (Chem. Ind. 1983, 478), have been described at least for aromatic rice (Oryza sativa L.). In this regard, pyrroline-5-carboxylic acid and methylglyoxal are named as potential precursors of 2-acetyl-1-pyrroline (J. Agric. Food Chem., 2008, 56 (16), pp 7399-7404).

The chemical synthesis of 2-acetyl-1-pyrroline is challenging. Though synthetic chemical routes to 2-acetyl-1-pyrroline have been described in the literature (J. Agric. Food Chem. 1998, 46, 616-619), 2-acetyl-1-pyrroline produced by such routes will suffer from its inherent instability and will therefore be of only limited use for food flavoring applications.

Technological attempts to stabilize the 2-acetyl-1-pyrroline are described in WO 2004/056202 A1, according to which a dispersible dry powder form is obtained.

Enhancement of the content of 2-acetyl-1-pyrroline may be achieved by precursor feeding of L-proline which was demonstrated in in vitro cultures of Pandanus amaryllifolius (see J. Sci. Food Agric. 2005, 85, 2527-2534).

The production of 2-acetyl-1-pyrroline via microbial cultures might also be feasible (Flavour and Fragrance Journal 2005, 20, 710-714).

Studies on the precursor content of 2-acetyl-1-pyrroline in pandan have been carried out by M. Jezussek (PhD thesis, Technical University of Munich, 2002). However, particular precursor compounds of 2-acetyl-1-pyrroline have not been identified in the study.

The Journal of the Science of Food and Agriculture 85, 1999-2004 (2005), describes a (optimized) process for the production of encapsulated pandan powder using spray-drying method.

The preferred object of the present invention was to provide a pandan extract article in powder form having improved flavor properties, in particular when combined with food products, in particular with regard to more intense and long lasting roasted notes and/or mouthfullness, especially in baked products, rice and fat based food products. More particularly, there is a strong need for a pandan extract comprising high amounts of (direct) precursors of (aroma-active) 2-acetyl-1-pyrroline.

A further object of the present invention is to provide a process of preparing such a pandan extract article.

Further objections underlying the present invention follow from the description below and the present patent claims.

According to a first aspect of the present invention, the underlying object is achieved by a pandan extract article in powder form, wherein the article comprises precursors of 2-acetyl-1-pyrroline and is obtainable by a process comprising the following steps:
providing pandan leaves or parts thereof,
contacting the pandan leaves or the parts thereof with water having a temperature in the range of from 5° C. to 40° C., preferably from 10 to 30° C., more preferably from 20 to 25° C., so that an aqueous extract of ingredients of pandan leaves is obtained,
removing water from the aqueous extract, in the presence or absence of a carrier material or other additives, wherein the water is removed by spray drying the aqueous extract using an inlet temperature in the range of from 140 to 170° C., preferably in the range of from 150 to 160° C., and an outlet temperature below 80° C., preferably an outlet temperature in the range of from 70 to 75° C.,
   so that the pandan extract article in powder form is obtained.

Pandan extract articles according to the present invention contain an advantageously high amount of precursors of 2-acetyl-1-pyrroline.

Without wishing to be bound by theory, it is believed that the pandan extract article according to the present invention comprises a relatively high amount of (unknown) precursors, since (free) amino acids, in particular (free) proline, glutamic acid and/or ornithin, originally contained in the pandan leaves or corresponding parts thereof are converted (during the process as described above) to precursors of 2-acetyl-1-pyrroline. However, as soon as the pandan extract article according to the invention is heated (e.g. in combination with a food product), in particular above 80° C., a strong and long lasting release of 2-acetyl-1-pyrroline is released, which is believed to result of the relatively high amount of precursors of 2-acetyl-1-pyrroline in the pandan extract article, which are—when activated by heat—converted into the (aroma-active) 2-acetyl-1-pyrroline.

A preferred pandan extract article according to the present invention (as described above), comprises less than 10 ppm, preferably 0.1 to 8 ppm, of (free) prolin, based on the total weight of the article.

A (further) preferred pandan extract article according to the present invention comprises 20 to 50 ppm of (free) glutamic acid, based on the total weight of the article.

A (further) preferred pandan extract article according to the present invention comprises 100 to 500 ppm of total free amino acids, based on the total weight of the article.

A (further) preferred Pandan extract article according to the present invention comprises 0.5 to 3 ppm of 3-Methyl-2(5H)-Furanon, based on the total weight of the article.

A (further) preferred Pandan extract article according to the present invention comprises 0.5 to 3 ppm of 2-Acetyl-1-Pyrroline, based on the total weight of the article.

Pandan extract articles according to the present invention are not known from the prior art. In particular, the Journal of the Science of Food and Agricultural 85, 1999-2004 (2005), does not disclose, neither explicitly nor implicitly, pandan extract articles according to the present invention, in particular pandan extract articles comprising the above amounts of (free) amino acids. It is believed that the temperature at which the pandan leaves or the parts thereof, respectively, are contacted with water (see above) as well as the used inlet and outlet temperatures when spray drying the aqueous extract (see above) are of particular importance.

Particularly preferred is a pandan extract article according to the present invention (as described above), wherein the water is removed from the aqueous extract in the presence of a carrier material, wherein the carrier material comprises an inclusion compound host material for including 2-acetyl-1-pyrroline. Preferably, the inclusion compound host material is also a host material for including above described (unknown) precursors of 2-acetyl-1-pyrroline therein.

It is particularly preferred that the carrier material comprises an inclusion compound host material wherein the inclusion compound host material is cyclodextrin, in particular alpha-cyclodextrin, beta-cyclodextrin or gamma-cyclodextrin, preferably beta-cyclodextrin.

The inclusion compound host material and the substance(s) to be included therein (here: at least 2-acetyl-1-pyrroline) form inclusion compounds, i.e. complexes in which one chemical compound (the inclusion compound “host” material) forms a cavity in which molecules of a second (or more) “guest” compound(s) (here: at least 2-acetyl-1-pyrroline) are located.

The presence of an inclusion compound host material as described above results in the advantage that such preferred pandan extract articles (as described above) provide a particularly prolonged release of 2-acetyl-1-pyrroline when heated, presumably due to the heat stability provided by the inclusion of 2-acetyl-1-pyrroline, which is believed to be (mainly) produced by heat-activated conversion from precursors of 2-acetyl-1-pyrroline (see above) to 2-acetyl-1-pyrroline.

According to an alternative or a further preferred solution according to the present invention, a pandan extract article (as described above), wherein the water is removed from the aqueous extract in the presence of a carrier material, wherein the carrier material (also) comprises maltodextrin, is particularly preferred.

A further aspect of the present invention relates to a process of preparing a pandan extract article in powder form, preferably a pandan extract article according to the present invention (as described above), the process comprising the following steps:
providing pandan leaves or parts thereof,
contacting the pandan leaves or the parts thereof with water having a temperature in the range of from 5° C. to 40° C., so that an aqueous extract of ingredients of pandan leaves is obtained,
removing water from the aqueous extract, in the presence or absence of a carrier material or other additives, wherein the water is removed by spray drying the aqueous extract using an inlet temperature in the range of from 140 to 170° C., preferably in the range of from 150 to 160° C., and an outlet temperature below 80° C., preferably an outlet temperature in the range of from 70 to 75° C.,
   so that the pandan extract article in powder form is obtained.

Preferred is a process as described above, wherein the water is removed from the aqueous extract in the presence of a carrier material, wherein the carrier material comprises an inclusion compound host material for including 2-acetyl-1-pyrroline and preferably precursors of 2-acetyl-1-pyrroline (see above) therein.

Further preferred is a process as described above, wherein the inclusion compound host material is cyclodextrin, in particular alpha-cyclodextrin, beta-cyclodextrin or gamma-cyclodextrin, preferably beta-cyclodextrin.

Particularly preferred is a process according to the present invention (as described above), wherein the water is removed from the aqueous extract in the presence of a carrier material, wherein the carrier material comprises maltodextrin.

A further aspect of the present invention relates to a method of imparting or enhancing organoleptic properties, the organoleptic properties being selected from the group consisting of mouthfullness and roasted notes, the method comprising the following steps:
providing a pandan extract article according to the present invention (as described above) or preparing a pandan extract article according to a process according to the present invention (as described above), and
combining the pandan extract article as provided or as prepared with a product.

Preferred is a method as described above, wherein the product is a consumable, preferably a food or beverage product.

Particularly preferred is a method according to the invention (as described above), the method comprising the additional step of
heating, preferably baking or cooking, the product obtained by combination with the pandan extract article as provided or as prepared in order to increase the release of 2-acetyl-1-pyrroline from the pandan extract.

By doing so, it is believed (without wishing to be bound by theory) that precursors of 2-acetyl-1-pyrroline, which are advantageously contained in high amounts in a pandan extract article according to the present invention, are converted to (aroma-active) 2-acetyl-1-pyrroline. That results in an advantageously prolonged and strong release of 2-acetyl-1-pyrroline.

If the pandan extract article is obtained by a process or a method, respectively, wherein the water is removed from the aqueous extract in the presence of a carrier material, wherein the carrier material comprises an inclusion compound host material (as described above), preferably cyclodextrin, strength and duration of the release of 2-acetyl-1-pyrroline is even more improved.

Also preferred is a method as described above, wherein the product is
rice or a rice-based product,
   or
a food product comprising one or more edible fats or oils, preferably a dairy or soy bean product.

A further aspect of the present invention relates to a method of masking an off-note of an edible fat or oil, the method comprising the following steps:
providing a pandan extract article according to the present invention (as described above) or preparing a pandan extract article according to a process according to the present invention (as described above),
combining the pandan extract article as provided or as prepared, respectively, with an edible fat or oil having an off-note or being prone to develop an off-note, so that said off-note is masked.

Particularly preferred is a method as described above, wherein the off-note is selected from the group consisting of cardboard notes, soy notes, beany notes, bitter notes, aldehydic notes, malty notes, astringent notes, rancid notes, musty notes and mouldy notes.

Further preferred is a method as described above, wherein the method comprises the additional step of
heating the product after combination with the pandan extract article as provided or as prepared so as to increase the release of 2-Acetyl-1-pyrroline from the pandan extract (cf. above).

A further aspect of the present invention relates to a method of making an edible food product, comprising the following steps:
providing a pandan extract article according to the present invention (as described above) or preparing a pandan extract article according to a method according to the present invention (as described above),
combining the pandan extract article as provided or as prepared, respectively, with a food product material,
   and
optionally heating, preferably baking or cooking, the food product material obtained by combination with the pandan extract article,
   in order to obtain the edible food product.

Preferred is a method as described above, wherein the edible food product is selected from the group consisting of baked products, cooked products, rice, rice-based products, and food products comprising one or more edible fats or oils. Further preferred food products are Coffee and coffee mix products, cereals, cookies, malt products, bread, snacks, fried noodles and soups.

The present invention thus also relates to an edible food product (as described above), comprising a pandan extract article according to the present invention (as described above) or a pandan extract article prepared according to a method according to the present invention (as described above).

A further aspect of the present invention relates to the use of a pandan extract article according to the present invention or prepared according to a method according to the present invention (as respectively described above) for
imparting or enhancing organoleptic properties, the organoleptic properties being selected from the group consisting of mouthfullness and roasted notes,
   and/or
masking off-notes of edible fats and oils.

Preferred embodiments and further aspects of the present invention emerge from the attached patent claims and the following example, the example is not being intended to limit the scope of the present invention.

### EXAMPLE

Production of a pandan extract article (according to the present invention):

At first, pandan leaves are cut about 5 to 10 cm off the top of stem. Then, the leaves are weighed (in required batch weight).

Afterwards the leaves are washed three times with water (tap water), keeping the several batches separate.

The leaves are grinded in the respective batches with water (ratio of dry leaves:water is approximately 1:4.5) at high speed on Stephan cutter for 4 minutes (afterwards: stand for 2 minutes). Then, the resulting mixtures are filtered through 400 micron bag filter to remove bulk of cut leaves and all filtrates are combined. The (combined) filtrate is acidified with citric acid (0.09 to 0.1% solution of 50% citric acid) to less than pH 4.5. The acidified filtrate is filtered through Seitz K100 filter using Filter press or Supradisc set up.

Afterwards, the resulting filtered pandan extract is spray dried,

using following spray dry emulsion recipe:
filtered pandan extract: 66.7 wt.-%
Maltodextrin DE 10-12: 33.3 wt.-%
(alternatively, for example 73 wt.-% of filtered pandan extract and 27 wt.-% of beta Cyclodextrin may be used)
   and following temperature settings:
inlet temperature: 160° C.
outlet temperature: 75° C.,
   so that a pandan extract article in powder form is obtained.

1. A powdered pandan extract composition comprising precursors of 2-acetyl-1-pyrroline obtainable by a process comprising:
providing pandan leaves or parts thereof,
contacting the pandan leaves or the parts thereof with water having a temperature in the range of from 5° C. to 40° C. so that an aqueous extract of ingredients of pandan leaves is obtained,
removing water from the aqueous extract, in the presence or absence of a carrier material or other additives, wherein the water is removed by spray drying the aqueous extract using an inlet temperature in the range of from 140 to 170° C. and an outlet temperature below 80° C.,
so that the powdered pandan extract composition is obtained.

2. The powdered pandan extract composition according to claim 1, wherein the composition comprises less than 10 ppm of prolin, based on the total weight of the article.

3. The powdered pandan extract composition according to claim 1, wherein the composition comprises 20 to 50 ppm of glutamic acid, based on the total weight of the article.

4. The powdered pandan extract composition according to claim 1, wherein the composition comprises 0.5 to 3 ppm of 2-Acetyl-1-Pyrroline, based on the total weight of the article.

5. The powdered pandan extract composition according to claim 1, wherein the composition comprises 100 to 500 ppm of total free amino acids, based on the total weight of the article.

6. The powdered pandan extract composition according to claim 1, wherein the composition comprises 0.5 to 3 ppm of 3-Methyl-2(5H)-Furanon, based on the total weight of the article.

7. The powdered pandan extract composition according to claim 1, wherein the water is removed from the aqueous extract in the presence of a carrier material, wherein the carrier material comprises an inclusion compound host material for including 2-acetyl-1-pyrroline.

8. The powdered pandan extract composition according to claim 7, wherein the inclusion compound host material is cyclodextrin.

9. The powdered pandan extract composition according to claim 1, wherein the water is removed from the aqueous extract in the presence of a carrier material, wherein the carrier material comprises maltodextrin.

10. A process for preparing a powdered pandan extract composition according to claim 1 comprising:
providing pandan leaves or parts thereof,
contacting the pandan leaves or the parts thereof with water having a temperature in the range of from 5° C. to 40° C., so that an aqueous extract of ingredients of pandan leaves is obtained,
removing water from the aqueous extract, in the presence or absence of a carrier material or other additives, wherein the water is removed by spray drying the aqueous extract using an inlet temperature in the range of from 140 to 170° C. and an outlet temperature below 80° C.,
so that the powdered pandan extract composition is obtained.

11. The process according to claim 10, wherein the water is removed from the aqueous extract in the presence of a carrier material, wherein the carrier material comprises an inclusion compound host material for including 2-acetyl-1-pyrroline.

12. The process according to claim 11, wherein the inclusion compound host material is cyclodextrin.

13. The process according to claim 10, wherein the water is removed from the aqueous extract in the presence of a carrier material, wherein the carrier material comprises maltodextrin.

14. A method of imparting or enhancing organoleptic properties, the organoleptic properties being selected from the group consisting of mouthfullness and roasted notes comprising:
providing a powdered pandan extract composition according to claim 1, and
combining the powdered pandan extract composition with a product.

15. The method according to claim 14, wherein the product is a consumable product.

16. The method according to claim 14, further comprising
heating the product combined with the powdered pandan extract composition to increase the release of 2-acetyl-1-pyrroline.

17. The method according to claim 14, wherein the product is
rice or a rice-based product,
or
a food product comprising one or more edible fats or oils.

18. A method of masking an off-note of an edible fat or oil comprising:
providing a powdered pandan extract composition according to claim 1, and
combining the powdered pandan extract composition with a product prepared with an edible fat or oil having an off-note or being prone to develop an off-note, so that said off-note is masked.

19. The method according to claim 18, wherein the off-note is selected from the group consisting of cardboard notes, soy notes, beany notes, bitter notes, aldehydic notes, malty notes, adstringent notes, rancid notes, musty notes and mouldy notes.

20. The method according to claim 18, further comprising
heating the product combined with the powdered pandan extract composition to increase the release of 2-acetyl-1-pyrroline.

21. A method of making an edible food product comprising:
providing a powdered pandan extract composition according to claim 1,
combining the powdered pandan extract composition with a food product material,
and
optionally heating the food product material combined with the powdered pandan extract composition
to obtain the edible food product.

22. The method according to claim 21, wherein the edible food product is selected from the group consisting of baked products, cooked products, rice, rice-based products, and food products comprising one or more edible fats or oils.

23. (canceled)
